(19) United States
(12) Patent Application Publication
(10) Pub. No.: US 2008/0299101 A1
(43) Pub. Date: Dec. 4, 2008

(54) Cancer Treatment Resistance and Agents Modulating Such Resistance

(75) Inventors: Isabelle Gourdier, Villejuif (FR); Arnaud Coquelle, Villejuif (FR); Bernard Dutrillaux, Paris (FR); Guido Kroemer, Antony (FR); Bernard Pau, Villejuif (FR)

Correspondence Address: FOLEY AND LARDNER LLP;SUITE 500, 3000 K STREET NW, WASHINGTON, DC, 20007, US

(73) Assignee: CENTRE NATIONAL DE LA RECHERCHE SCIENTIFIQUE (CNRS INSTITUT PASTEUR

(21) Appl. No.: 11/571,798

(22) PCT Filed: Jul. 8, 2005
(86) PCT No.: PCT/IB2005/002629

§ 371 Date: Apr. 4, 2008

(30) Foreign Application Priority Data
Jul. 9, 2004 (CA) 2 474 442

(51) Int. Cl. A61K 38/43; C12Q 1/68; C12N 9/00; A61P 31/00; A61K 31/28

(52) U.S. Cl. 424/94.1; 435/6; 435/183; 514/492

(57) ABSTRACT

The present invention concerns a method for detecting in vitro resistance of cancer cells to a treatment by detecting in the cancer cells supernumerary nucleolar organizer regions (NOR), whereby identification of the presence of supernumerary NOR in the cancer cells is indicative of treatment resistance. The present invention also concerns a method for defining the capacity of a candidate agent to modulate NOR genes number of cancer cells by contacting the cancer cells with a candidate agent for a time sufficient to permit modulation of NOR genes number; and observing whether NOR genes number increases or decreases in said cancer cells. The present invention also provides a candidate agent that modulates NOR genes number of cancer cells obtained by the method of the invention and a composition for treating and/or preventing a cancer in a patient, comprising said candidate agent. Such a candidate agent decreases the NOR genes number in said cancer cells.

### FIELD OF THE INVENTION

The present invention relates to the field of cancer treatment of patients, and more specifically to those cancers that are resistant to treatment, such as an oxaliplatin treatment.

### BACKGROUND OF THE INVENTION

The chimiotherapeutic treatments of cancers, in spite of the availability of active antitumor molecules like oxaliplatin, see their efficacy very limited by the frequent appearance of cancer cells resistance to the cytotoxic effects of the drugs, used alone or in combination. The reduction of this resistance is thus a major stake for our health and pharmaceutical industries.

The Applicants have already described in WO 03/107006 a method for detecting in vitro resistance of cancer cells to an oxaliplatin treatment. Such a method particularly consists in measuring mitochondrial apoptosis of cancer cells being treated or capable of being treated or to be treated with oxaliplatin.

However, according to the Applicant's knowledge, there is no predictive marker to a response to a cancer treatment, such as an oxaliplatin treatment.

Therefore, there is still a need for new methods for detecting in vitro resistance of cancer cells to a treatment and compositions to circumvent such cancer treatment resistance.

### SUMMARY OF THE INVENTION

The present invention relates to methods that satisfy the above mentioned need.

More particularly, one object of the invention concerns a method for detecting in vitro resistance of cancer cells to a treatment, comprising the step of detecting in said 30 cancer cells supernumerary nucleolar organizer regions (NOR), whereby identification of the presence of supernumerary NOR in said cancer cells is indicative of treatment resistance.

Another object of the invention is to provide a method for defining the capacity of a candidate agent to modulate NOR genes number of cancer cells, comprising the steps of:

a. contacting the cancer cells with a candidate agent for a time sufficient to permit modulation of NOR genes number, and

b. observing whether NOR genes number increases or decreases in said cancer cells.

Furthermore, the present invention has also for an object a candidate agent that modulates NOR genes number of cancer cells obtained by the method as defined above, its use in composition and method for treating and/or preventing a cancer in a patient.

Another object of the present invention is a method for treating and/or preventing a cancer in a patient comprising the step of administering to said patient a therapeutically effective amount of a composition comprising an agent that modulates the NOR genes number of cancer cells, optionally in association with an active antitumor molecule, such as oxaliplatin.

Other objects and advantages of the present invention will be apparent upon reading the following non-restrictive detailed description, made with reference to the accompanying drawings.

### BRIEF DESCRIPTION OF THE DRAWINGS

FIG. 1 shows an example of karyotype for HCT116/S.

FIG. 2 shows an example of karyotype for HCT116/R2. Arrow shows the p21+.

FIG. 3 shows silver staining of Ag NOR in HCT116/S. Arrows show the active NOR (positive). No amplification is detected.

FIG. 4 shows silver staining of Ag NOR in HCT116/R2. Arrows show the active NOR (positive). Large arrow shows the amplification.

FIG. 5 shows silver staining of Ag NOR in HCT116/Rev1. Arrows show the active NOR (positive). No amplification is present.

FIG. 6 shows silver staining of Ag NOR in HCT116/R2. Arrows show the active NOR (positive). Large arrows show the amplification.

FIG. 7 shows another silver staining of Ag NOR in HCT116/R2. Arrows show the active NOR (positive). Large arrows show the amplification.

FIG. 8A shows silver staining of Ag NOR in SW48/S. Arrows show the active NOR (positive). No amplification is present.

FIG. 8B shows silver staining of Ag NOR in SW48/R1. Arrows show the active NOR (positive). Large arrows show the amplification

FIG. 9 shows silver staining of Ag NOR in SW48/R2. Arrows show the active NOR (positive). Large arrows show the amplification.

FIG. 10 shows silver staining of Ag NOR in SW480/S. Arrows show the active NOR (positive). No amplification is present.

FIG. 11A shows silver staining of Ag NOR in SW480/R. Arrows show the active NOR (positive). Large arrows show the amplification.

FIG. 11B shows silver staining of Ag NOR in SW480/R. Arrows show the active NOR (positive). Large arrows show the amplification.

FIGS. 12 to 14 show fluorescence in situ hybridisation (FISH) of ribosomal DNA in HCT116/Rev2.

FIGS. 15 and 16 show fluorescence in situ hybridisation (FISH) of ribosomal DNA in HCT116/S.

FIG. 17 shows the location og AgNOR.

FIG. 18 shows the localisation of supernumerary AgNOR in HCT 116 model system.

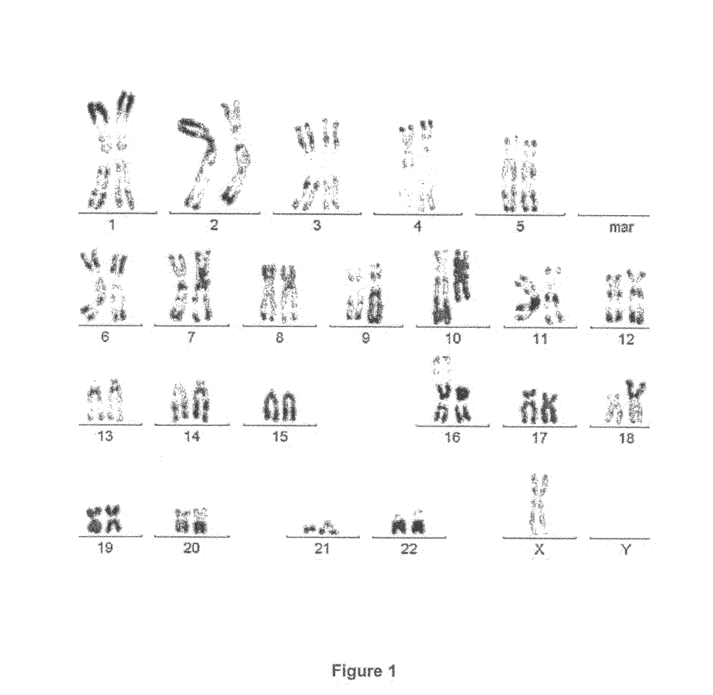

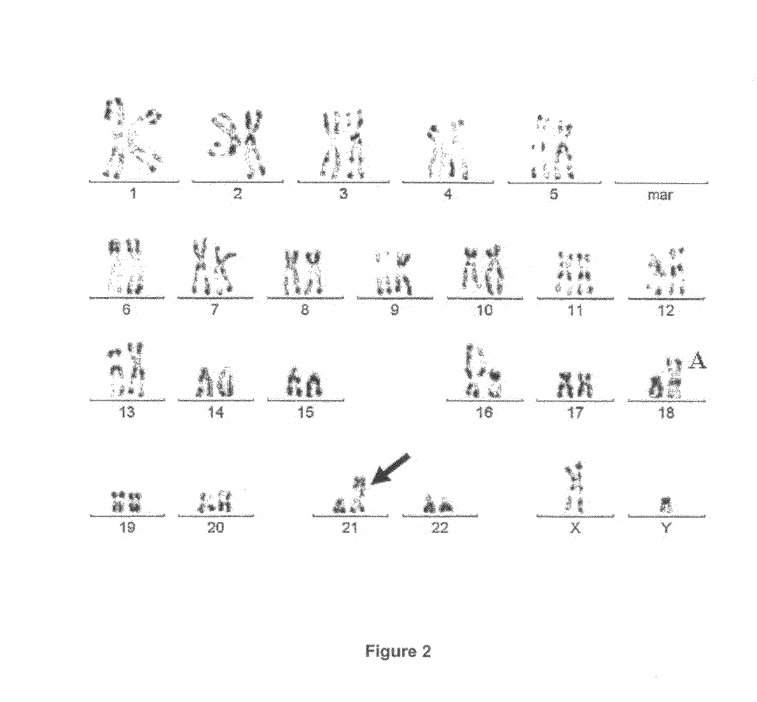

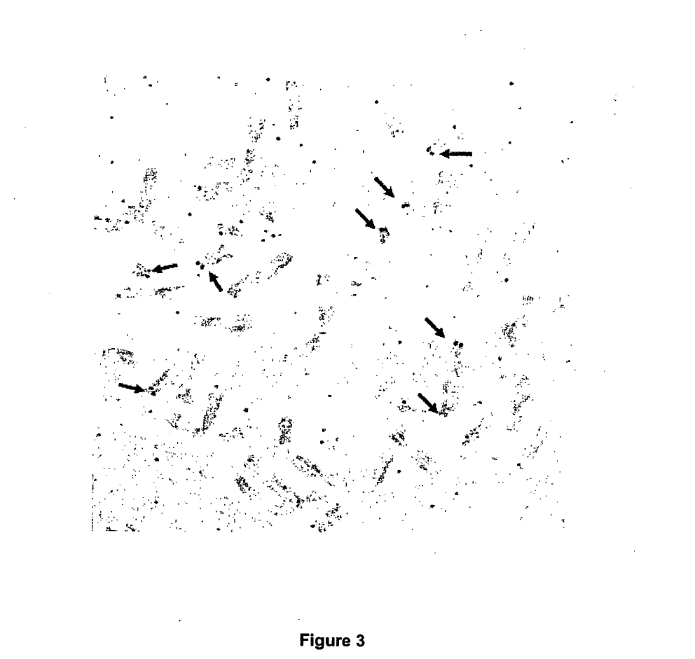

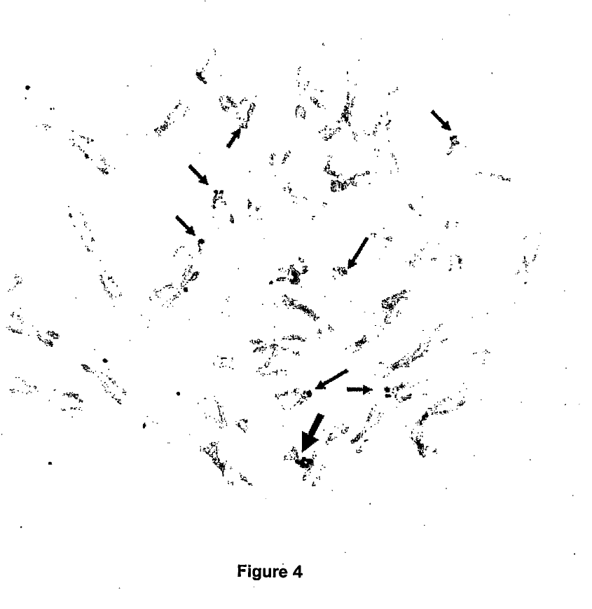

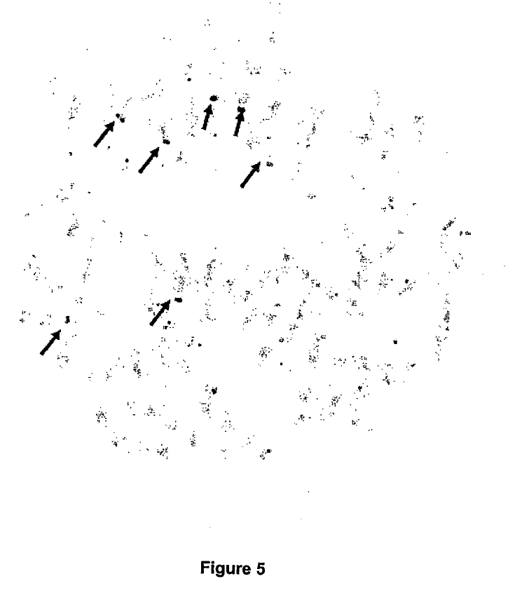

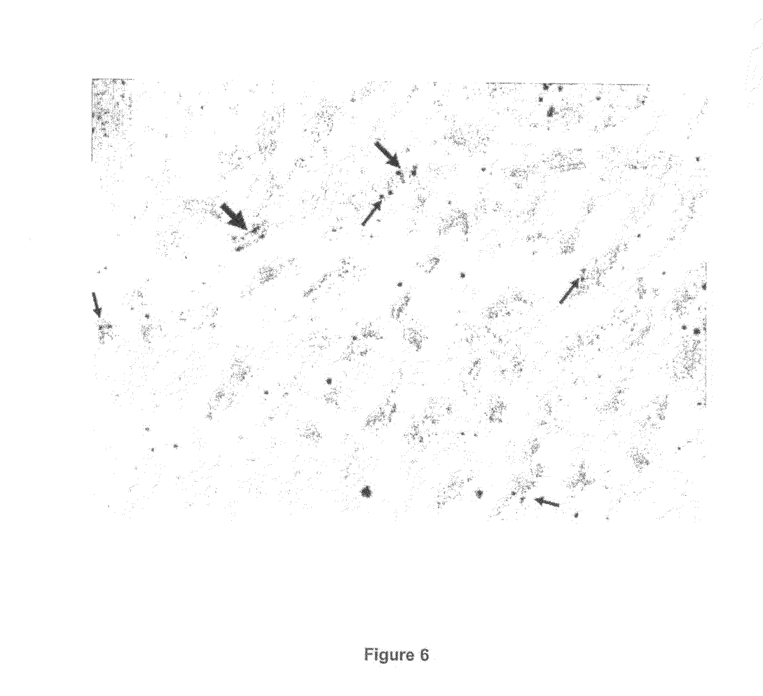

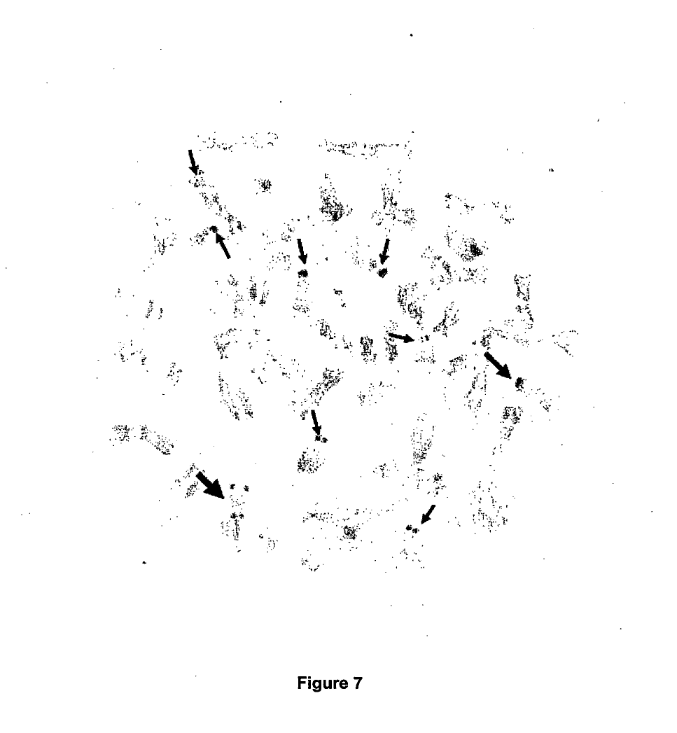

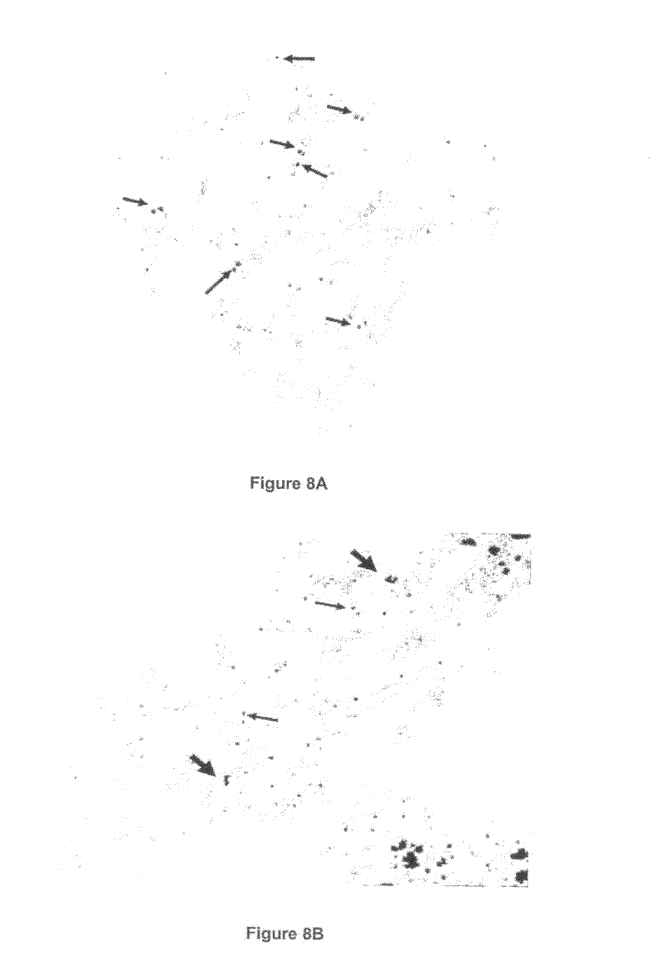

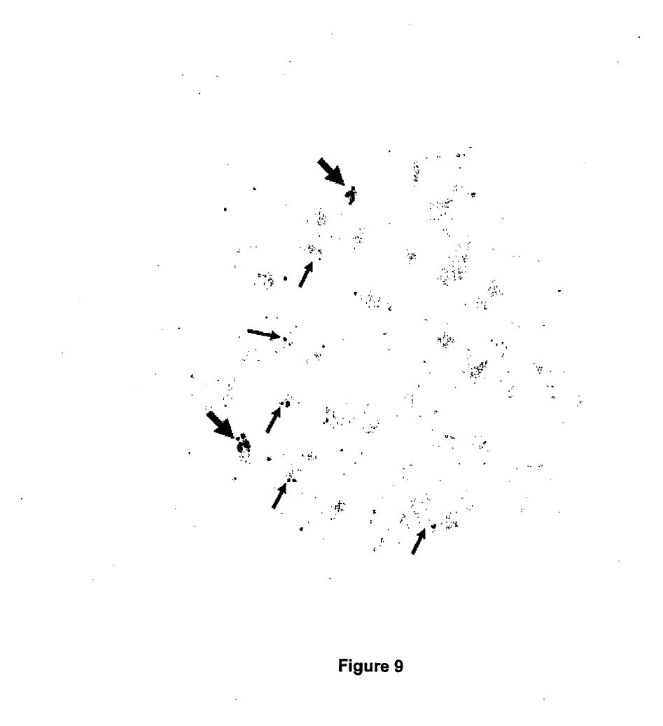

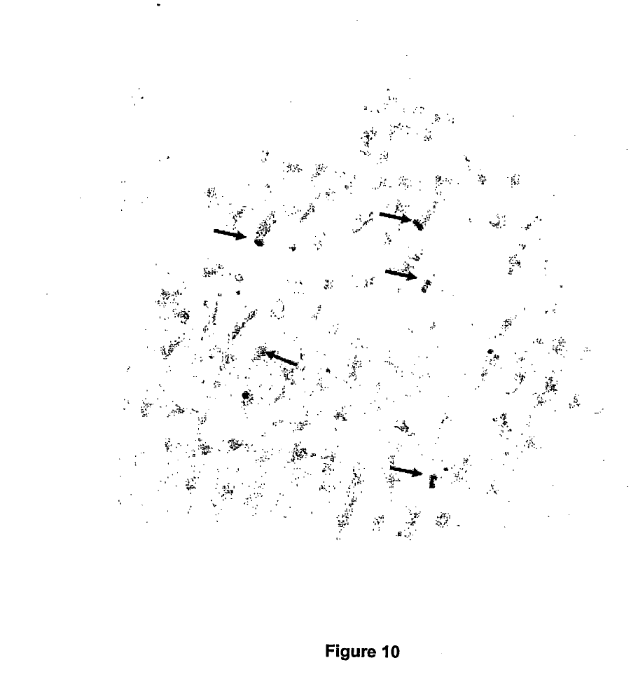

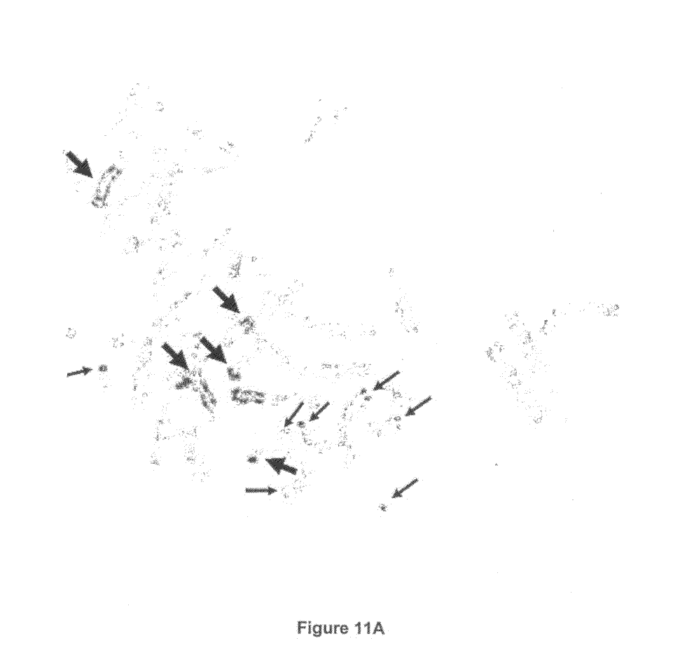

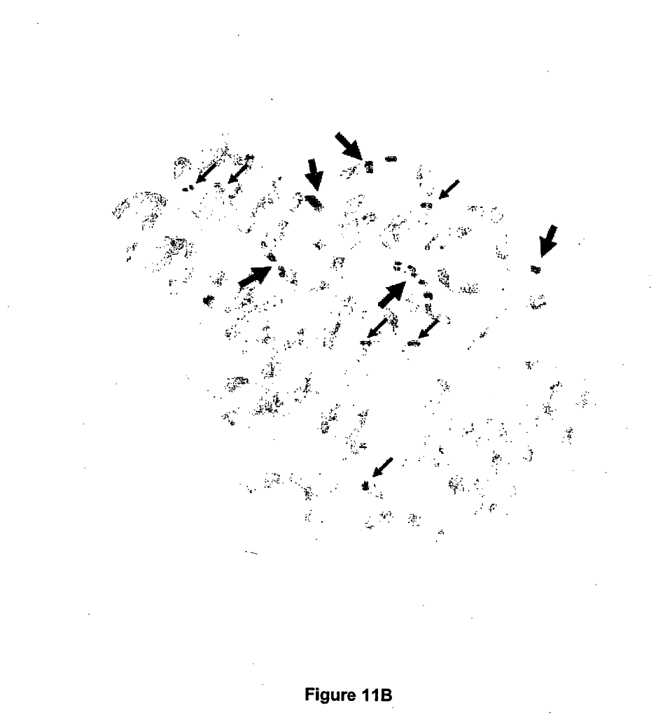

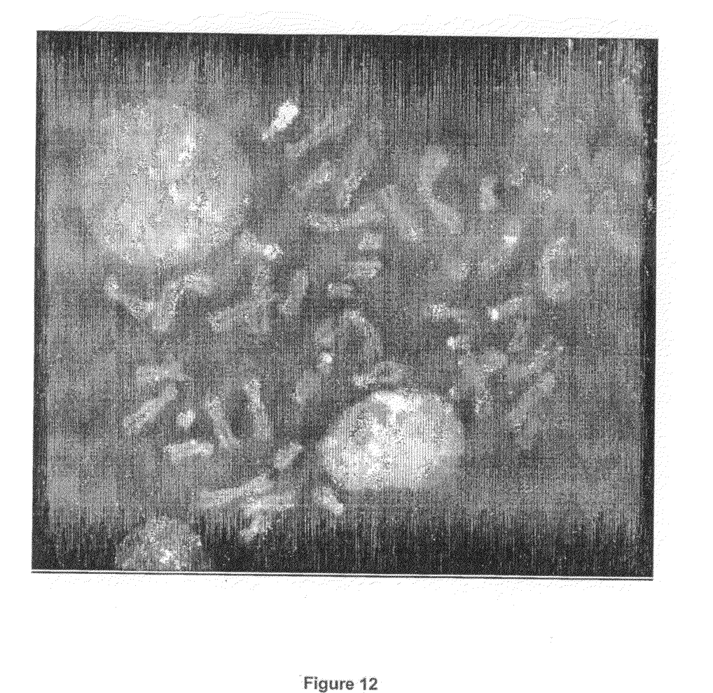

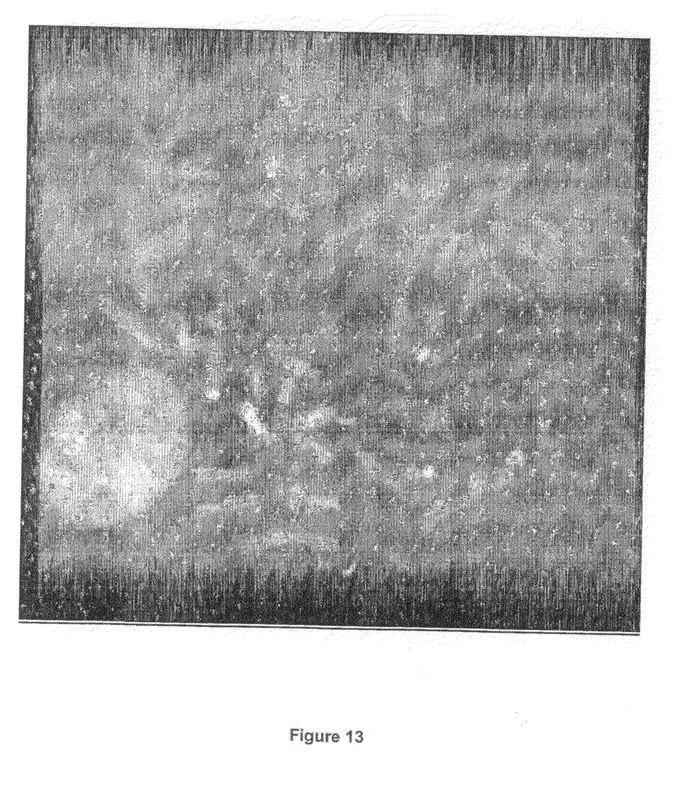

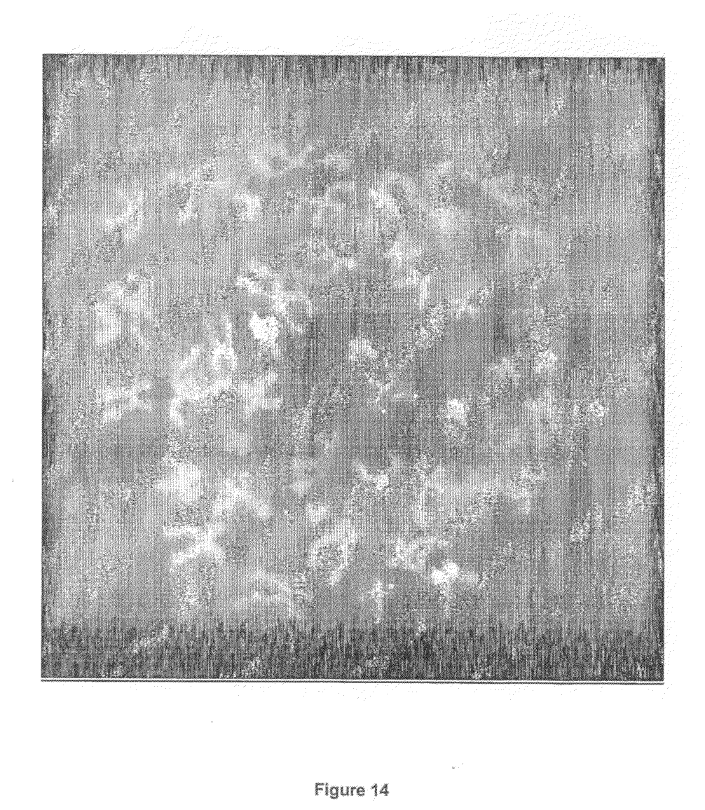

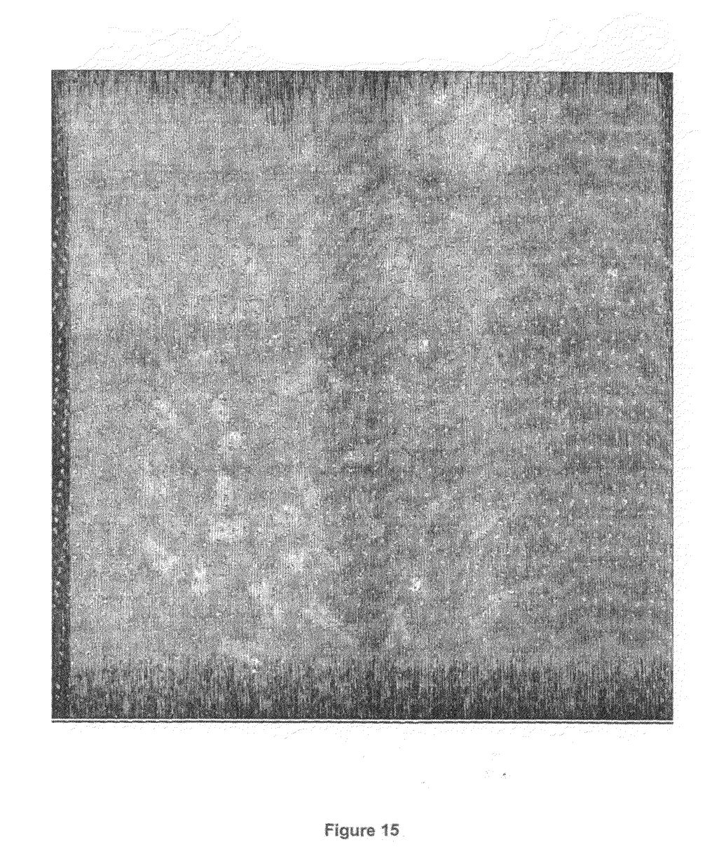

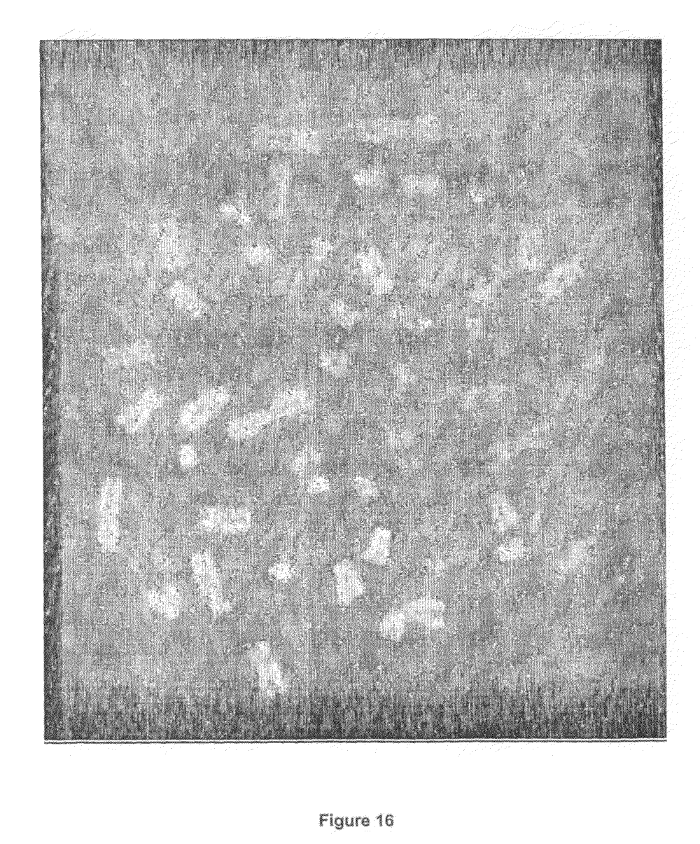

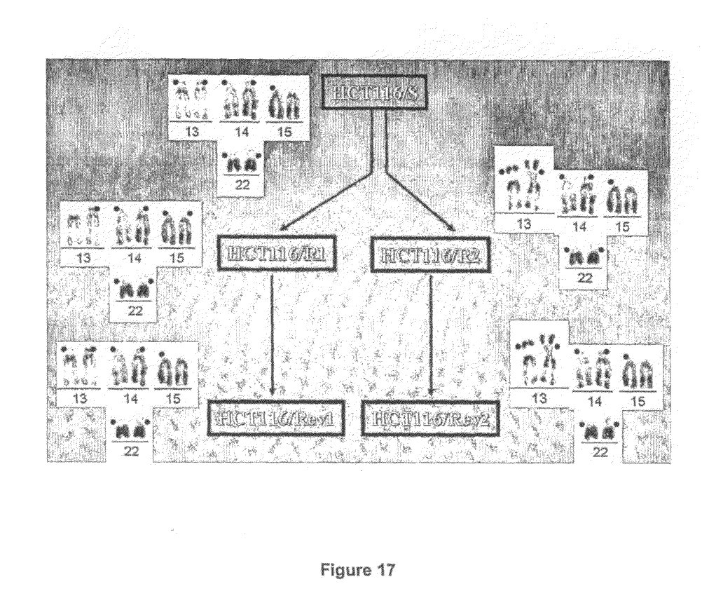

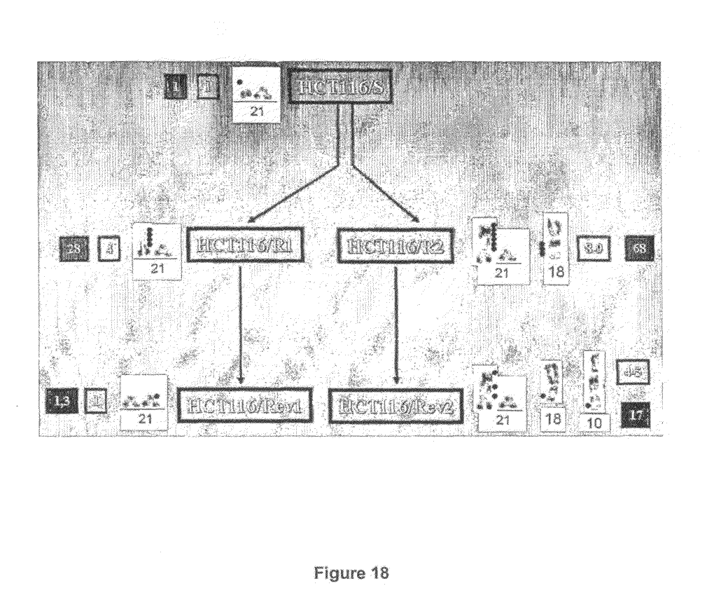

### DETAILED DESCRIPTION OF THE INVENTION

The present invention relates to the field of cancer treatment of patients, and more specifically to those cancers, such as some colorectal cancers, that are resistant to treatment, such as an oxaliplatin treatment.

Despite the fact that it is known in the art that the analysis of the nucleolar organizer regions (NOR) finds a practical application in tumor pathology (see for review Pich, A et al. 2000. Micron 31: 133-141 and Ofner, D. 2000. Micron 31: 161-164), the inventors of the present application have surprisingly found a correlation between oxaliplatin resistance and apparition of supernumerary NOR. Consequently, the present invention relates to a method for detecting in vitro resistance of cancer cells to a treatment and to a method for defining the capacity of a candidate agent to modulate NOR genes number of cancer cells and the use of such candidate agent in composition and method for treating and/or preventing a cancer in a patient.

A non-exhaustive list of cancer cells that are within the scope of the invention are those being treated or capable of being treated or to be treated with an anti-cancer molecule such as oxaliplatin, and more specifically cancer cells such as those selected from the group consisting of colorectal cancer cells, ovarian cancer cells, germinal cancer cells, lung cancer cells, digestive tract cancer cells, prostatic cancer cells, pancreatic cancer cells, stomach cancer cells and small intestine cancer cells.

### 1. Methods of Detecting

As a first embodiment, the present application provides a method for detecting in vitro resistance of cancer cells to a treatment, comprising the step of detecting in said cancer cells supernumerary nucleolar organizer regions (NOR), whereby identification of the presence of supernumerary NOR in said cancer cells is indicative of treatment resistance. It will be understood that such a method is preferably used to detect oxaliplatin resistant cancer cells. In a particular embodiment, the cancer is a colorectal cancer.

As used herein, the expression “supernumerary NOR” refers to amplification of NOR genes in a cancer cell type. In other words, the number of NOR genes of said cancer cell type is substantially higher that the NOR genes number of the cell type.

The expression “substantially higher” refers to an increase of about two folds, and more preferably of about four folds or higher, in the number of NOR genes.

Another embodiment of the invention concerns a method for defining the capacity of a candidate agent to modulate NOR genes number of cancer cells, comprising the steps of:

a. contacting the cancer cells with a candidate agent for a time sufficient to permit modulation of NOR genes number; and

b. observing whether NOR genes number increases or decreases in said cancer cells.

Particularly, such a method allows defining the capacity of a candidate molecule to modulate resistance of cancer cells to a treatment, such as an oxaliplatin treatment for example.

Modulation of NOR genes number is defined as the capacity of agents or molecules of the invention to either increase or decrease the NOR activity either by increasing or decreasing the number of NOR genes in cells, or for instance, by upregulating or downregulating NOR at the level of transcription or translation, in a cell.

In the above methods, and more particularly concerning the step of detecting in said cancer cells supernumerary NOR as recited in the method of the first embodiment and step b) of the method of the second embodiment, said steps are preferably achieved by any known detecting method to one skilled in the art, such as silver-staining and fluorescence in situ hybridisation (FISH) methods.

In a related embodiment, the present invention is concerned with a candidate agent that modulates NOR genes number of cancer cells preferably obtained by the above mentioned method, wherein the candidate agent decreases the NOR genes number in said cancer cells. Preferably, the candidate agent of the invention is capable of regulating the ribosomic RNA level in said cancer cells. For instance, the candidate agent may consist of an inhibitor of RNA transcription such as Actinomycin D, or of a ribonuclease such as alpha-sarcine.

### 2. Methods of Treatment And Compositions

Agents that modulate, preferably decrease, the NOR genes number, such as the candidate agents obtained by the method of the invention, may be used in many ways in the treatment of cancers, and especially in combination with an anti-cancer molecule such as oxaliplatin.

In another embodiment, the present invention relates to a composition for preventing or treating cancer in a patient. Such a composition comprises a candidate agent as defined above in combination with an anti-cancer molecule, and an acceptable carrier for treating and/or preventing a cancer in an animal.

It will be understood that the anti-cancer molecule preferably contemplated by the present invention is oxaliplatin, but any other platin salts, such as cis-platin or carboplatin, and optionally in association with another agents such as 5-FU (5-fluorouracile) for example, are within the scope of the present invention.

As used herein, the term “treating” refers to a process by which the symptoms of a cancer are alleviated or completely eliminated. As used herein, the term “preventing” refers to a process by which symptoms of a cancer are obstructed or delayed.

As used herein, the expression “an acceptable carrier” means a vehicle for containing the composition of the invention that can be injected into a host without adverse effects. Suitable carriers known in the art include, but are not limited to, gold particles, sterile water, saline, glucose, dextrose, or buffered solutions. Carriers may include auxiliary agents including, but not limited to, diluents, stabilizers (i. e., sugars and amino acids), preservatives, wetting agents, emulsifying agents, pH buffering agents, viscosity enhancing additives, colors and the like.

Further agents can be added to the composition of the invention. For instance, the composition of the invention may also comprise agents such as drugs, immunostimulants (such as α-interferon, β-interferon, γ-interferon, granulocyte macrophage colony stimulator factor (GM-CSF), macrophage colony stimulator factor (M-CSF), interleukin 2 (IL2), interleukin 12 (IL12), and CpG oligonucleotides), antioxidants, surfactants, flavoring agents, volatile oils, buffering agents, dispersants, propellants, and preservatives. For preparing such compositions, methods well known in the art may be used.

The amount of anti-cancer molecule (eg. oxaliplatin) and candidate agent of the invention is preferably a therapeutically effective amount. A therapeutically effective amount of oxaliplatin and candidate agent of the invention is that amount necessary to allow the same to perform their anti-cancer role without causing, overly negative effects in the host to which the composition is administered. The exact amount of oxaliplatin and candidate agent of the invention to be used and the composition to be administered will vary according to factors such as the type of cancer being treated, the mode of administration, as well as the other ingredients in the composition.

The composition of the invention may be given to a host through various routes of administration. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. The composition of the invention may also be formulated as creams, ointments, lotions, gels, drops, suppositories, sprays, liquids or powders for topical administration. It may also be administered into the airways of a subject by way of a pressurized aerosol dispenser, a nasal sprayer, a nebulizer, a metered dose inhaler, a dry powder inhaler, or a capsule. Suitable dosages will vary, depending upon factors such as the amount of each of the components in the composition, the desired effect (short or long term), the route of administration, the age and the weight of the host to be treated. Any other methods well known in the art may be used for administering the composition of the invention.

In a further embodiment, the present invention provides a method for treating and/or preventing a cancer in a patient comprising the step of administering to said patient a therapeutical effective amount of a composition comprising an agent that modulates, preferably decreases, the NOR genes number, such as the composition of the invention.

In another aspect, the present invention is directed to the use of a composition comprising an agent that modulates, preferably decreases, the NOR genes number, such as the composition of the invention, for the manufacture of a drug intended to treat and/or to prevent a cancer in a patient.

In a particular embodiment, the cancer is a colorectal cancer. As used herein, the “ribosomal DNA” contemplated by the present invention has advantageously a nucleotide sequence as set forth in NCBI Accession number U13369.

### EXAMPLES

The present invention will be more readily understood by referring to the following examples. These examples are illustrative of the wide range of applicability of the present invention and is not intended to limit its scope. Modifications and variations can be made therein without departing from the spirit and scope of the invention. Although any methods and materials similar or equivalent to those described herein can be used in the practice for testing of the present invention, the preferred methods and materials are described.

### Introduction

To study oxaliplatin resistance, the inventors have generated four cellular models using four colorectal cancer cell lines HCT116, SW48, SW480 and SW620 (obtained from ATCC). Each cellular model is composed of one sensitive clone (representative of the parental cell line), one or two resistant clones isolated by exposure to increasing oxaliplatin concentrations. For the HCT116 cellular model, “total or partial revertant” clones are obtained from maintaining resistant clones in absence of oxaliplatin during several months. The inventors investigated genetic modifications associated with oxaliplatin resistance by caryotyping first the HCT116 cellular model. This investigation has led to define a strict correlation between oxaliplatin resistance and apparition of supernumerary NOR (nucleolar organizer regions where ribosomal genes are clustered). Reciprocally, reversion of the resistant phenotype is correlated with loss of supernumerary NOR, as detected in revertant clones. Subsequent analysis of active NOR (by silver coloration of metaphasis) in all cellular models allowed the inventors to reveal a systematic NOR amplification in resistant clones, a return to initial number of NOR in total revertant clones and an intermediary number of NOR in partial revertants. Identification of the modulation of NOR number as strictly correlated with oxaliplatin resistance is particularly pertinent because it was found in the four cellular models which present different genetic backgrounds.

So this discovery could be quite universal. The potential applications of this gene amplification concern early diagnostics of oxaliplatin resistance on tumor pieces as well as drug modulation of resistance focused on the effects of NOR amplification. Identification of NOR amplification on paraffin-embedded tissues sections from colorectal cancer patients is being settled. Two methodologies are under investigation in the present applicaton: silver-staining and FISH using ribosomic probe (sequence located on the ribosomal RNA). This last technique (FISH) has been validated on the HCT116 cellular model. In addition, experiments designed to pharmacologically modulate the activity of oxaliplatin using actinomycin D were performed. Some results suggest that an actinomycin D preincubation of the cells at moderately toxic concentrations lead to an synergistic effect on oxaliplatin activity.

### Example 1

### Identification of NOR Gene Amplification On Paraffin-Embedded Tissues Sections

### Slide Preparation

Exponentially growing cells from the different clones were treated with colcemid (100 ng/ml) or nocodazole (10 μM) for 2-4 h. Cells are:
1) trypsinated, centrifugated at 1200 rpm for 5 min.,
2) rinsed with PBS, centrifugated at 1200 rpm for 5 min.,
3) incubated 10 mn −37° C. in 0.0075 M KCL/10% fetal calf serum, centrifugated at 1200 rpm for 5 min.,
4) fixated in 3 volume absolute ethanol/1 volume Acetic acid, centrifugated at 1200 rpm for 5 min. (this step: 2 to 5 times),
5) spread on slides and used for cytological studies.

### FISH Protocol For rDNA

FISH is performed essentially as described.

### 1) RNAse Treatment

1 h at 37° C. in 2SSC (ph=7)—RnAse (final concentration: 100 μg/ml)

10 min in 2SCC Room Temperature (RT)

10 min in 2SCC RT

10 min in 50% ethanol RT

10 min in 75% ethanol RT

10 min in 100% ethanol RT

air dry

### 2) Probe Preparation

for each slide prepare: 2 μl of probe+8 μl of hybmix. Labelling of the probe according to supplier recommendation (1 μg of DNA is labelled, according to supplier's recommendation)

### 3) Slides Denaturation

2 min in 70% formamide/2SSC. 70° C.

1 min in 2SSC. 4° C.

5 min in 50% ethanol. 4° C.

5 min in 75% ethanol. 4° C.

5 min in 100% ethanol. 4° C.

air dry

### 4) Probe Denaturation

10 min at 96-100° C.

on ice

### 5) Hybridization

add 10 pi of denatured probe per slide. Apply glass coverslip and seal.

Incubate overnight at 37° C. in a humidified chamber

### 6) Post-Hybridization Wash

remove coverslip

5 min in 50% formamide/2SSC. 38° C.

5 min in 50% formamide/2SSC. 38° C.

5 min in 2SSC. 38° C.

5 min in 2SSC. 38° C.

Depending on the protocol of probe labelling (indirect or direct), probes are revealed with classical antibodies (for example: goat anti-biotin/anti-goat FITC) or not. Slides are then mounted in an antifading preparation with propidium iodide (Vectashield; Biosys S. A.).

### Silver Staining Protocol For AgNOR Quantitation

200 μl of staining solution per slide (staining solution: 0.01 g gelatin/ml, 0.25 g AgNO₃/ml, 0.5% formic acid): incubate at 70° C. for 90-120 seconds.
Pour off the solution and rinse the slides with distilled water.
Giemsa staining: 4 minutes in 1.5% giemsa.

| | Oxaliplatin resistance | Number of |
|---|---|---|
| Line | (fold/parental line) | surnumerar AgNOR |
| HCT116/S (parental) | 1 | 1 |
| HCT116/R1 | 28 | 4 |
| HCT116/R2 | 68 | 8-9 |
| HCT116/Rev1 | 1 | 1 |
| HCT116/Rev2 | 17 | 4-5 |

| | Oxaliplatin resistance | Amplification of |
|---|---|---|
| Line | (fold/parental line) | AgNOR/parental |
| HCT116/S (parental) | 1 | no |
| HCT116/R1 | 28 | yes |
| HCT116/R2 | 68 | yes |
| HCT116/Rev1 | 1 | no |
| HCT116/Rev2 | 17 | yes |
| SW48/S | 1 | no |
| SW48/R1 | 26 | yes |
| SW48/R2 | 73 | yes |
| SW480/S | 1 | no |
| SW480/R | 63 | yes |
| SW620/S | 1 | no |
| SW620/R | 22 | yes |

### Example 2

### Preliminary Experiment of Modulation of Oxaliplatin Cytotoxicity Using Actinomycin D Preincubation

The following example concerns preliminary preincubation results of cells with actinomycin D which shows a synergy effect of the actinomycin D with oxaliplatin.

HCT116/S

| | | | Cytotoxicity of | | Cytotoxicity of | | Cytotoxicity of |
|---|---|---|---|---|---|---|---|
| | Cytotoxicity | Cytotoxicity of | oxaliplatin* in | Cytotoxicity of | oxaliplatin* in | Cytotoxicity of | oxaliplatin* in |
| Oxaliplatin | of oxaliplatin | oxali + actD | presence of actD | oxali + actD | presence of actD | oxali + actD | presence of actD |
| (μM) | (%) | 0.04 nM (%) | 0.04 nM (%) | 0.12 nM (%) | 0.12 nM (%) | 0.36 nM (%) | 0.36 nM (%) |
| 0 | 0 | 9.1 | 0 | 27.6 | 0 | 85.4 | 0 |
| 0.16 | 18.5 | 31.9 | 25.1 | 52.6 | 34.5 | 91.4 | 41.9 |
| 0.31 | 40.3 | 55.2 | 50.7 | 71.5 | 60.6 | 91.5 | 42.6 |
| 0.62 | 61.6 | 74.7 | 72.2 | 83.2 | 76.8 | 93.3 | 54.7 |
| 1.25 | 74.4 | 79.5 | 77.5 | 85.6 | 80.1 | 92.5 | 49.3 |
| 2.5 | 76.7 | 79.4 | 77.3 | 76.8 | 76.8 | 90.4 | 35.1 |

HCT116/R1

| | | | Cytotoxicity of | | Cytotoxicity of | | Cytotoxicity of |
|---|---|---|---|---|---|---|---|
| | Cytotoxicity | Cytotoxicity of | oxaliplatin* in | Cytotoxicity of | oxaliplatin* in | Cytotoxicity of | oxaliplatin* in |
| Oxaliplatin | of oxaliplatin | oxali + actD | presence of actD | oxali + actD | presence of actD | oxali + actD | presence of actD |
| (μM) | (%) | 0.04 nM (%) | 0.04 nM (%) | 0.12 nM (%) | 0.12 nM (%) | 0.36 nM (%) | 0.36 nM (%) |
| 0 | 0 | 17.3 | 0 | 18 | 0 | 37.3 | 0 |
| 1.25 | 17.2 | 27.4 | 12.2 | 36.5 | 22.6 | 52.9 | 24.9 |
| 2.5 | 17.6 | 37.3 | 24.2 | 47.3 | 35.7 | 66.3 | 46.3 |
| 5 | 47 | 53.2 | 44.1 | 56.9 | 47.4 | 75.4 | 60.8 |
| 10 | 67 | 68.7 | 62.2 | 71.7 | 65.5 | 78.9 | 66.4 |
| 20 | 65.8 | 71.1 | 65.1 | 74.3 | 68.7 | 80.5 | 68.9 |

HCT116/R2

| | | | Cytotoxicity of | | Cytotoxicity of | | Cytotoxicity of |
|---|---|---|---|---|---|---|---|
| | Cytotoxicity of | Cytotoxicity of | oxaliplatin* in | Cytotoxicity of | oxaliplatin* in | Cytotoxicity of | oxaliplatin* in |
| Oxaliplatin | oxaliplatin | oxali + actD | presence of actD | oxali + actD | presence of actD | oxali + actD | presence of actD |
| (μM) | (%) | 0.04 nM (%) | 0.04 nM (%) | 0.12 nM (%) | 0.12 nM (%) | 0.36 nM (%) | 0.36 nM (%) |
| 0 | 0 | 20.6 | 0 | 17.7 | 0 | 21.6 | 0 |
| 2.5 | 24.7 | 27.2 | 8.3 | 32.6 | 18.1 | 52.8 | 39.8 |
| 5 | 31.7 | 45.4 | 31.2 | 49.2 | 38.3 | 66.6 | 57.4 |
| 10 | 55.6 | 66.5 | 57.8 | 69.9 | 63.4 | 74.4 | 67.4 |
| 20 | 70.4 | 71.3 | 63.9 | 73.5 | 67.8 | 80.9 | 75.6 |
| 40 | 74.2 | 79.8 | 74.6 | 78 | 72.3 | 83.3 | 78.7 |

*Cytotoxicity of oxaliplatin in presence of actinomycine D (actD) is calculated as follow: 100 − ((viability of oxaliplatin + actD/viability of actD) × 100)

Cytotoxicity assays were performed by seeding 4000 cells per well in 96 well plates. After a 24 h rest for cell attachment, cells were preincubated for 48 h with actinomycin D before incubation for 48 h of oxaliplatin. Cytotoxicity measurements were carried out using the WST-1 colorimetric assay (Boehringer Mannheim) according to the manufacturer's recommendation.

### Example 3

### Study of the Expression Levels of Ribosomal RNA In Different Cellular Models

For the two types of ribosomal RNA studied (18S RNA and 28S RNA), the inventors observed an overexpression of ribosomal RNA by the resistant cell lines HCT116_R1, HCT116_R2 compared to the oxaliplatin sensitive HCT116_S. The results obtained for a single RNA extraction demonstrates an overexpression of 18S and 28S RNA for the SW620/R line and an overexpression of 18S RNA for the SW480/R line. These results confirm and complete the upstream amplification previously observed at the gene level (RNA-NOR) which codes for ribosomal RNA. This increase is even more significant since the transcription products of the NOR genes are, in every cell, very abundant and largely in majority compared to all other RNAs. The observation that the inventors have already related, of an increase of transcriptionally active RNA-NOR (revealed by silver staining, see Figures) has supplied a strong argument for predicting an increase in ribosomal RNA. The present results prove that the amplification of RNA-NORs leads in fine to an increase in cellular content of ribosomal RNA. As evoked hereabove, ribosomal RNA is in majority and represents about 40 to 60% of the transcription activity of a cell as well as 80% of the total cellular RNA content. This finding implies that the overexpression of ribosomal RNA, which appears in relation with oxaliplatin resistance, must have an important biological significance for the resistant phenotype since it is not inconsequential for the sub-cellular organisation. This overexpression of ribosomal RNA, which occurs as an extension of genetic amplification, constitutes a new diagnostic element, and an element of pharmacological intervention.

| | 18S Ribosomal RNA | 28S Ribosomal RNA |
|---|---|---|
| HCT116_R1/S | 1.6 ± 0.22 | 2.1 ± 0.33 |
| | (1.3 ± 0.28; 1.4 ± 0.36; | (1.8 ± 0.39; 2.5 ± 0.97; |
| | 1.7 ± 0.11) | 2.2 ± 0.45) |
| HCT116_R2/S | 1.8 ± 0.38 | 1.6 ± 0.42 |
| | (1.6 ± 0.23; 2.1 ± 0.48) | (1.3 ± 0.30; 1.9 ± 0.59) |
| SW48_R1/S | 1.4 ± 0.30 | 1.1 ± 0.39 |
| SW480_R/S | 1.5 ± 0.01 | 0.9 ± 0.22 |
| SW620_R/S | 1.9 ± 0.57 | 1.9 ± 0.65 |

The levels of expression were measured by quantitative PCR in real time (LightCycler System—Roche) from total RNA extractions retro-transcribed into complementary DNA. The values correspond to the ratio of the levels of expression between the resistant cell lines and the sensitive cell line of reference (average, followed by standard deviation). For the SW48, SW480 and SW620 lines, the average and the standard deviation were obtained by 3 or 4 independent PCR experiments measured in real time, from one same RNA extraction. For the HCT116 cell line, each average and standard deviation was determined from 3 or 4 independent PCR experiments measured in real time, from one same RNA extraction; the values in italic correspond to the average of the expression ratios obtained for different RNA extractions.

1. Method for detecting in vitro resistance of cancer cells to a treatment, comprising the step of detecting in said cancer cells supernumerary nucleolar organizer regions (NOR), whereby identification of the presence of supernumerary NOR in said cancer cells is indicative of treatment resistance.

2. The method of claim 1, wherein said treatment resistance consists of an oxaliplatin treatment resistance.

3. Method for defining the capacity of a candidate agent to modulate NOR genes number of cancer cells, comprising the steps of:
a. contacting the cancer cells with a candidate agent for a time sufficient to permit modulation of NOR genes number; and
b. detecting an increase or decrease in the number of NOR genes in said cancer cells.

4. The method of claim 1, wherein the cancer cells are cancer cells being treated or capable of being treated or to be treated with oxaliplatin.

5. The method of claim 1, wherein the cancer cells are selected from the group consisting of colorectal cancer cells, ovarian cancer cells, germinal cancer cells, lung cancer cells, digestive tract cancer cells, prostatic cancer cells, pancreatic cancer cells, stomach cancer cells and small intestine cancer cells.

6. The method of claim 1, wherein the step of detecting in said cancer cells supernumerary NOR is achieved by a detecting method selected from the group consisting of silver-staining and fluorescence in situ hybridisation (FISH).

7. A candidate agent that modulates NOR genes number of cancer cells obtained by the method of claim 3, wherein the candidate agent decreases the NOR genes number in said cancer cells.

8. The candidate agent of claim 7, wherein said candidate agent regulates the ribosomic RNA level in said cancer cells.

9. The candidate agent of claim 8, wherein said candidate agent is an inhibitor of RNA transcription.

10. The candidate agent of claim 8, wherein said candidate agent is a ribonuclease.

11. A composition for treating and/or preventing a cancer in a patient, comprising the candidate agent of claim 7 in combination with an anti-cancer molecule, and a pharmaceutically acceptable carrier.

12. The composition according to claim 11, wherein the anti-cancer molecule is oxaliplatin.

13. A method for treating and/or preventing a cancer in a patient comprising the step of administering to said patient a therapeutically effective amount of a composition comprising an agent that modulates the NOR genes number of cancer cells.

14. (canceled)

15. The method of claim 13, wherein said composition is the composition of claim 11.

16. The method of claim 13, wherein said composition decreases the NOR genes number of cancer cells.

17. The method of claim 13, wherein the cancer is a cancer being treated or capable of being treated or to be treated with oxalip.

18. The method of claim 13, wherein the cancer is selected from the group consisting of colorectal cancer, ovarian cancer, germinal cancer, lung cancer, digestive tract cancer, prostate cancer, pancreatic cancer, stomach cancer and small intestine cancer.

19. The method of claim 3, wherein the step of detecting an increase or decrease in the number of NOR genes is achieved by a detecting method selected from the group consisting of silver-staining and fluorescence in situ hybridisation (FISH).

20. The candidate agent of claim 9, wherein said inhibitor of RNA transcription is Actinomycin D.

21. The candidate agent of claim 10, wherein said ribonuclease is alpha-sarcine.

22. The composition according to claim 11, wherein said candidate agent regulates the ribosomic RNA level in said cancer cells.

23. The composition according to claim 22, wherein said candidate agent is an inhibitor of RNA transcription.

24. The composition according to claim 23, wherein said candidate agent is Actinomycin D.

25. The composition according to claim 22, wherein said candidate agent is a ribonuclease.

26. The composition according to claim 25, wherein said candidate agent is alpha-sarcine.

27. A method for manufacturing a composition for the treatment and/or prevention of cancer comprising the step of combining an agent that modulates the NOR genes number of cancer cells with a pharmaceutically acceptable carrier.

28. The method of claim 27, wherein said composition is the composition of claim 11.

29. The method of claim 27, wherein said composition decreases the NOR genes number of cancer cells.

30. The method of claim 27, wherein the cancer is a cancer being treated or capable of being treated or to be treated with oxalip.

31. The method of claim 27, wherein the cancer is selected from the group consisting of colorectal cancer, ovarian cancer, germinal cancer, lung cancer, digestive tract cancer, prostate cancer, pancreatic cancer, stomach cancer and small intestine cancer.
